SEQUENCE LISTING (5 sequences)

SEQ ID NO: 1 - DNA, 1584 bp, synthetic construct
  source 1..1584: mol_type = other DNA; note = PPAR-g protein
atggattggccttttgcaagaaatagacaaaatatcagtgtgaatgacagcaaacccctattccatgctgtcatgggtgaaactctgggagattctcttattgacccagaaagcgatgccttcgacacgctgtctgcaaacatttcacaagaggtgaccatggttgacaccgagatgccgttttggcccaccaactttgggatcagctctgtggacctgtcggtgatggacgaccactcccactcctttgacatcaagcccttcaccactgttgatttctccagcatttccactccacactatgaagacatcccattcccgagagctgatccaatggttgcagattataagtacgacctgaagctccaggactaccaaagtgccatcaaagtggagcctgtgtccccgccttattattctgaaaagactcagctgtacaacaaacctcacgaagagccttccaactccctcatggcaattgaatgccgtgtctgtggggataaagcctcggggttccactatggagttcatgcttgtgaaggatgcaagggtttcttccggaggactatcagattgaagcttatttatgataggtgcgatcttaactgtcggatccacaaaaaaagtagaaataaatgtcagtactgtcggtttcagaaatgccttgctgtggggatgtctcataacgccatcaggtttgggcggatgccacaggctgagaaggagaagctgttggcagagatctccagtgatatcgaccagctgaacccagagtctgctgacctccgcgccctggcaaagcacttgtatgactcatacataaagtccttcccgctgaccaaagcaaaggcgagggcgatcttgacaggaaagaccacagacaaatcaccctttgtcatctatgacatgaattccttaatgatgggagaagataaaatcaagttcaaacacatcacccccctgcaggagcagagcaaagaggtggccattcgcatctttcaggggtgtcagtttcgctccgtggaggccgtgcaggagatcacagagtatgccaagaacatccctgggtttgtaaaccttgacctgaatgaccaagtaactctcctaaagtatggcgtccacgagatcatttacaccatgctggcctccttgatgaataaagacggggtcctcatctccgagggccaaggattcatgacaagggagtttctcaagagcctgagaaagccctttggtgactttatggagcccaagttcgagtttgctgtgaagttcaacgcactggaattagatgacagcgacctggcgatatttatagctgtcattattctcagtggagaccgcccaggtttgctgaatgtgaagcccatcgaggacattcaagacaatttgctgcaagccttggagctgcagctcaagttgaaccacccagagtcctcccagctctttgctaagctgctccagaaaatgacagacctcagacagattgtgacagagcacgtgcagctgctgcaagtaataaagaagacagagacggacatgagccttcacccactcctacaggaaatatacaaggacttgtac

SEQ ID NO: 2 - DNA, 1065 bp, synthetic construct
  source 1..1065: mol_type = other DNA; note = CEPBa protein
atggagtcggccgacttctacgaggcggagccgcggcccccgatgagcagccacctccagagccccccgcacgcgcccagcagcgccgccttcggctttccccggggcgcgggccccgcgcagcccccagccccacctgccgccccggagccgctgggcggcatctgcgaacacgagacgtccatcgacatcagcgcctacatcgacccggccgccttcaacgacgagttcctggccgacctgtttcagcacagccggcagcaagagaaggccaaggcggccgcggcccccgcgggaggcggcggcgactttgactacccgggcgcccccgtgggccccggcggcgccgtcatgcccgggggggctcacggcccccctcccagctacggctgcgcggcggccggctacctggacggcaggctggagcccctgtacgagcgcgtcggggcgccggcgctgcggccgctagtgatcaagcaggagccgcgcgaggaggacgagtcgaagcagctggcgctggccggcctctttccctaccagccaccgccgccaccgccaccaccgcattcacaccctccgcccgcacacctggccgccccgcacttgcagttccagatcgcgcactgcggccagaccaccatgcacctgcagcccggccaccccacgccgccgcccacgcccgtgcccagcccgcacccagcgcccgcgcttggcgccgctggcctgccaggccccggcggcgctctcaaggggctggccccagcgcaccccgacctgcgcgcgggcggcggcggcggcgcaggcaaagccaagaagtcggtagacaagaacagcaacgagtaccgggtgcggcgtgagcgcaacaacatcgcggtgcgcaagagccgggacaaggccaagcagcgcaacgtggagactcaacagaaggtgctggagctgaccagtgacaatgaccgcctgcgcaagcgggtggaacaactgagccgcgaactggacacgctgcggggcatcttccgccagctgcccgagagctccctggtcaaggccatgggcaactgcgcttga

SEQ ID NO: 3 - DNA, 66 bp, synthetic construct
  source 1..66: mol_type = other DNA; note = GSG-P2A_linker
ggatctggagcaacaaacttctcactactcaaacaagcaggtgacgtggaggagaatcccggcccc

SEQ ID NO: 4 - DNA, 2727 bp, synthetic construct
  source 1..2727: mol_type = other DNA; note = PPARG-P2A-CEBPA - construct
atggattggccttttgcaagaaatagacaaaatatcagtgtgaatgacagcaaacccctattccatgctgtcatgggtgaaactctgggagattctcttattgacccagaaagcgatgccttcgacacgctgtctgcaaacatttcacaagaggtgaccatggttgacaccgagatgccgttttggcccaccaactttgggatcagctctgtggacctgtcggtgatggacgaccactcccactcctttgacatcaagcccttcaccactgttgatttctccagcatttccactccacactatgaagacatcccattcccgagagctgatccaatggttgcagattataagtacgacctgaagctccaggactaccaaagtgccatcaaagtggagcctgtgtccccgccttattattctgaaaagactcagctgtacaacaaacctcacgaagagccttccaactccctcatggcaattgaatgccgtgtctgtggggataaagcctcggggttccactatggagttcatgcttgtgaaggatgcaagggtttcttccggaggactatcagattgaagcttatttatgataggtgcgatcttaactgtcggatccacaaaaaaagtagaaataaatgtcagtactgtcggtttcagaaatgccttgctgtggggatgtctcataacgccatcaggtttgggcggatgccacaggctgagaaggagaagctgttggcagagatctccagtgatatcgaccagctgaacccagagtctgctgacctccgcgccctggcaaagcacttgtatgactcatacataaagtccttcccgctgaccaaagcaaaggcgagggcgatcttgacaggaaagaccacagacaaatcaccctttgtcatctatgacatgaattccttaatgatgggagaagataaaatcaagttcaaacacatcacccccctgcaggagcagagcaaagaggtggccattcgcatctttcaggggtgtcagtttcgctccgtggaggccgtgcaggagatcacagagtatgccaagaacatccctgggtttgtaaaccttgacctgaatgaccaagtaactctcctaaagtatggcgtccacgagatcatttacaccatgctggcctccttgatgaataaagacggggtcctcatctccgagggccaaggattcatgacaagggagtttctcaagagcctgagaaagccctttggtgactttatggagcccaagttcgagtttgctgtgaagttcaacgcactggaattagatgacagcgacctggcgatatttatagctgtcattattctcagtggagaccgcccaggtttgctgaatgtgaagcccatcgaggacattcaagacaatttgctgcaagccttggagctgcagctcaagttgaaccacccagagtcctcccagctctttgctaagctgctccagaaaatgacagacctcagacagattgtgacagagcacgtgcagctgctgcaagtaataaagaagacagagacggacatgagccttcacccactcctacaggaaatatacaaggacttgtacggatctggagcaacaaacttctcactactcaaacaagcaggtgacgtggaggagaatcccggccccggttccctccggatggagtcggccgacttctacgaggcggagccgcggcccccgatgagcagccacctccagagccccccgcacgcgcccagcagcgccgccttcggctttccccggggcgcgggccccgcgcagcccccagccccacctgccgccccggagccgctgggcggcatctgcgaacacgagacgtccatcgacatcagcgcctacatcgacccggccgccttcaacgacgagttcctggccgacctgtttcagcacagccggcagcaagagaaggccaaggcggccgcggcccccgcgggaggcggcggcgactttgactacccgggcgcccccgtgggccccggcggcgccgtcatgcccgggggggctcacggcccccctcccagctacggctgcgcggcggccggctacctggacggcaggctggagcccctgtacgagcgcgtcggggcgccggcgctgcggccgctagtgatcaagcaggagccgcgcgaggaggacgagtcgaagcagctggcgctggccggcctctttccctaccagccaccgccgccaccgccaccaccgcattcacaccctccgcccgcacacctggccgccccgcacttgcagttccagatcgcgcactgcggccagaccaccatgcacctgcagcccggccaccccacgccgccgcccacgcccgtgcccagcccgcacccagcgcccgcgcttggcgccgctggcctgccaggccccggcggcgctctcaaggggctggccccagcgcaccccgacctgcgcgcgggcggcggcggcggcgcaggcaaagccaagaagtcggtagacaagaacagcaacgagtaccgggtgcggcgtgagcgcaacaacatcgcggtgcgcaagagccgggacaaggccaagcagcgcaacgtggagactcaacagaaggtgctggagctgaccagtgacaatgaccgcctgcgcaagcgggtggaacaactgagccgcgaactggacacgctgcggggcatcttccgccagctgcccgagagctccctggtcaaggccatgggcaactgcgcttga

SEQ ID NO: 5 - DNA, 7751 bp, synthetic construct
  source 1..7751: mol_type = other DNA; note = pMI014_PPARG-P2A-CEBPA_opti-ox-aavs1
aattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctcactgctgagggcctcaggccagtccatcctcgtcttgtctctttcgccctgctctttccccaccttgagcgctcttaaccagcctggcccgtgccacctctactctgccatcgaatgctgccccactttctcgagtccgccacttctcccagcttcaccggtacccactgtttcccctagtccaggcaggtaccactttccctgagcgtcctcctcctctcttctgggcctgtgctgcttcttttcccgctctctggcctgggccgtttcttcggccagcccccgagccttccatgccctttccttcaggtttctgctcttcatccttggtctctgccatctgttgccatgtaagggtgctctttcctgagccatcgccctcaaggcgctctgctcctcaagtggatgcttccctcgcctggctcacctcctgctctctctcctgcccccttcacctgcgtgccctcctcattctccctctgtgccacctctggccttgcactgtaggctctctcttggggatgtttctctttctccacacacttctctttcactctgtcctcttgctttgtgtgggcctgcagcgttacccttttttctgggcacactcagagcaccctcctctttctggttctgggccacctgtctgtcctcgggtcatcttgctctctctgcctggatgccctcctgtggctttgggcagcttctccctccttcagagtgcaccgccaaagcttctgacctcttctcttcctcccacagggcctcgagagatctggcagcggagagggcagaggaagtcttctaacatgcggtgacgtggaggagaatcccggccctaggctcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttagccaccatggattggccttttgcaagaaatagacaaaatatcagtgtgaatgacagcaaacccctattccatgctgtcatgggtgaaactctgggagattctcttattgacccagaaagcgatgccttcgacacgctgtctgcaaacatttcacaagaggtgaccatggttgacaccgagatgccgttttggcccaccaactttgggatcagctctgtggacctgtcggtgatggacgaccactcccactcctttgacatcaagcccttcaccactgttgatttctccagcatttccactccacactatgaagacatcccattcccgagagctgatccaatggttgcagattataagtacgacctgaagctccaggactaccaaagtgccatcaaagtggagcctgtgtccccgccttattattctgaaaagactcagctgtacaacaaacctcacgaagagccttccaactccctcatggcaattgaatgccgtgtctgtggggataaagcctcggggttccactatggagttcatgcttgtgaaggatgcaagggtttcttccggaggactatcagattgaagcttatttatgataggtgcgatcttaactgtcggatccacaaaaaaagtagaaataaatgtcagtactgtcggtttcagaaatgccttgctgtggggatgtctcataacgccatcaggtttgggcggatgccacaggctgagaaggagaagctgttggcagagatctccagtgatatcgaccagctgaacccagagtctgctgacctccgcgccctggcaaagcacttgtatgactcatacataaagtccttcccgctgaccaaagcaaaggcgagggcgatcttgacaggaaagaccacagacaaatcaccctttgtcatctatgacatgaattccttaatgatgggagaagataaaatcaagttcaaacacatcacccccctgcaggagcagagcaaagaggtggccattcgcatctttcaggggtgtcagtttcgctccgtggaggccgtgcaggagatcacagagtatgccaagaacatccctgggtttgtaaaccttgacctgaatgaccaagtaactctcctaaagtatggcgtccacgagatcatttacaccatgctggcctccttgatgaataaagacggggtcctcatctccgagggccaaggattcatgacaagggagtttctcaagagcctgagaaagccctttggtgactttatggagcccaagttcgagtttgctgtgaagttcaacgcactggaattagatgacagcgacctggcgatatttatagctgtcattattctcagtggagaccgcccaggtttgctgaatgtgaagcccatcgaggacattcaagacaatttgctgcaagccttggagctgcagctcaagttgaaccacccagagtcctcccagctctttgctaagctgctccagaaaatgacagacctcagacagattgtgacagagcacgtgcagctgctgcaagtaataaagaagacagagacggacatgagccttcacccactcctacaggaaatatacaaggacttgtacggatctggagcaacaaacttctcactactcaaacaagcaggtgacgtggaggagaatcccggccccggttccctccggatggagtcggccgacttctacgaggcggagccgcggcccccgatgagcagccacctccagagccccccgcacgcgcccagcagcgccgccttcggctttccccggggcgcgggccccgcgcagcccccagccccacctgccgccccggagccgctgggcggcatctgcgaacacgagacgtccatcgacatcagcgcctacatcgacccggccgccttcaacgacgagttcctggccgacctgtttcagcacagccggcagcaagagaaggccaaggcggccgcggcccccgcgggaggcggcggcgactttgactacccgggcgcccccgtgggccccggcggcgccgtcatgcccgggggggctcacggcccccctcccagctacggctgcgcggcggccggctacctggacggcaggctggagcccctgtacgagcgcgtcggggcgccggcgctgcggccgctagtgatcaagcaggagccgcgcgaggaggacgagtcgaagcagctggcgctggccggcctctttccctaccagccaccgccgccaccgccaccaccgcattcacaccctccgcccgcacacctggccgccccgcacttgcagttccagatcgcgcactgcggccagaccaccatgcacctgcagcccggccaccccacgccgccgcccacgcccgtgcccagcccgcacccagcgcccgcgcttggcgccgctggcctgccaggccccggcggcgctctcaaggggctggccccagcgcaccccgacctgcgcgcgggcggcggcggcggcgcaggcaaagccaagaagtcggtagacaagaacagcaacgagtaccgggtgcggcgtgagcgcaacaacatcgcggtgcgcaagagccgggacaaggccaagcagcgcaacgtggagactcaacagaaggtgctggagctgaccagtgacaatgaccgcctgcgcaagcgggtggaacaactgagccgcgaactggacacgctgcggggcatcttccgccagctgcccgagagctccctggtcaaggccatgggcaactgcgcttgatgctagcgcggccgcatcgataagcttgtcgacgatatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccttgacagtactcttaagtcgacttaggggacctagagccctgctaggtcctctctctccacaacctgggcccccaaacctttccaaaacaccttgctttctgcctccattggtcttgtgttccagagccagagtcactatatgtcccagaaccaggattccctctggttctgagggcttttatcgcatcccctgcctggctgcagtgggtctttggggacaggccacagaagagcctctactcctccctctgtccccgaggctgtctccctcccagtcttcccagctcaggccagtccccaggcctctcttccctgccagagcccgtcaggttcggttactttggggcccagagaggaccctgtgaaggaagcgtgggtaggggcacgggaatggggaggatgcctgaagaggcccccttagccagaagaggagcagaagaggagcaggtacccagaagaggagcagttcagggaaatagaagagtcccgagcaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatcctttta

(19) United States
(12) Patent Application Publication
(10) Pub. No.: US 2026/0357211 A1
(43) Pub. Date: Nov. 12, 2026

(54) Adipocyte maturation

(71) Applicant: MEATABLE B.V., Leiden (NL)

(72) Inventors: Ruud OUT, Leiden (NL); Kyle Bill QUINNEY, Leiden (NL); Sara PALACIOS ORTERA, Leiden (NL)

(73) Assignee: MEATABLE B.V., Leiden (NL)

(21) Appl. No.: 19/121,633

(22) PCT Filed: Oct. 20, 2023
(86) PCT No.: PCT/EP2023/079366

§ 371 Date: Apr. 16, 2025

(30) Foreign Application Priority Data
Oct. 21, 2022 (EP) 22203081.9

(51) Int. Cl. C12N 5/077; A23L 13/00; C07K 14/47; C07K 14/705; C12N 5/0735; C12N 5/074; C12N 15/63; C12N 15/85

(52) U.S. Cl. CPC C12N 5/0653; A23L 13/00; C07K 14/4702; C07K 14/70567; C12N 5/0606; C12N 5/0696; C12N 15/635; C12N 15/85; C12N 2506/02; C12N 2506/45; C12N 2830/003; C12N 2830/006; C12N 2830/34

(57) ABSTRACT

The present invention relates to pluripotent stem cell comprising an expression construct for expression of a PPAR-γ protein and an expression construct for expression of a CEBPα protein. The invention further provides for methods of producing adipocytes comprising the pluripotent stem cells and for foodstuff comprising the adipocytes or pluripotent stem cells.

### FIELD OF THE INVENTION

The present invention relates to modified pluripotent cells and to methods of differentiating said cell into adipocytes.

### BACKGROUND OF THE INVENTION

According to the most recent United Nations estimations, the current world population is 7.9 billion in July 2022 [https://www.worldometers.info/es/poblacion-mundial/#ref-1] and it is expected to reach 10 billion around the year 2056. This increase will be heterogeneously distributed around the globe, with nine countries covering half the projected growth of the global population in the next 30 years, including India, Nigeria, Pakistan, Egypt, and the United States of America. Population and economic growth are major drivers of increased meat consumption. According to the United Nations Food and Agricultural Organization (FAO, https://www.oecd-ilibrary.org/agriculture-and-food/oecd-fao-agricultural-outlook-2022-2031_f1b0b29c-en), an estimated growth of 15% in global meat consumption is projected by 2031. On the other hand, the correlation between income growth and higher meat consumption is clearly demonstrable at lower income rates but once consumers reach an adequate standard of living, they become more sensitive to environmental, ethical, and animal welfare and health concerns.

For this reason, there is a growing interest in finding alternative protein sources which ideally will be sustainable and will contain the nutrients normally provided by meat in the human diet. Cultured meat arises as another alternative to traditional animal agriculture that aims to produce the skeletal muscle and adipose tissues that normally comprise animal meats, except using in vitro tissue and biological engineering techniques. Despite efforts to develop robust protocols for scalable generation of animal cell types from easily accessible and renewable sources, the differentiation of animal (pluripotent) stem cells into specific cell types often remains cumbersome, lengthy, and difficult to reproduce and/or has not been established yet.

Additionally, to date, plant-based and cultured meat alternatives have focused mostly on mimicking the muscle component of meat. However, fat is also a crucial component of meat, contributing to sensory/flavor, textural attributes and palatability (Zhang, Shu, et al. “DNA polymorphisms in bovine fatty acid synthase are associated with beef fatty acid composition 1.” Animal genetics 39.1 (2008): 62-70.).

Modified cell lines that can be differentiated to adipocytes have been previously described by Tontonoz et al (Cell, Vol. 79, 1147-1156-30 Dec. 1994) wherein a retroviral expression system is used to co-express PPARγ and CEBPα into a fibroblast cell line upon which spontaneous differentiation to adipocytes was observed. US2012219530 describes lentivirally transduced human pluripotent cells that have a differentiation efficiency of about 20%. However these protocols have several limitation including heterogeneous adipocyte degree of maturation, lack of scalable process, are not food safe and have long cultivation times of up to 28 days.

Accordingly there remains a need in the art for the production and culturing of mature adipocytes that are suitable for human consumption and that can be produced in a scalable and cost effective manner.

### SUMMARY OF THE INVENTION

In a first aspect, the invention relates to a pluripotent stem cell comprising:
i) an expression construct for expression of a transcriptional regulator protein inserted into a first genetic safe harbour site;
ii) an expression construct for expression of a PPAR-γ protein, wherein the coding sequence for the PPAR-γ protein is operably linked to an inducible promoter; and,
iii) an expression construct for expression of a CEBPα protein, wherein the coding sequence for the CEBPα protein is operably linked to an inducible promoter;
wherein the expression constructs of ii) and iii) are inserted into at least one further genetic safe harbour site that is not the first genetic safe harbour site,
and wherein the inducible promotor is regulated by the transcriptional regulator protein.

In certain embodiments of the invention, the expression constructs of ii) and iii) are both inserted into a second genetic safe harbour site that is different from the first genetic safe harbour site. Preferably, said first and further genomic safe harbour sites are selected from any two of the hROSA26 locus, the AAVS1 locus, the CLYBL gene or the CCR5 gene, preferably wherein the genetic safe harbour site are hROSA26 locus and the AAVS1 locus.

In certain embodiments of the invention, the cell is selected from the group consisting of embryonic stem cells, induced pluripotent stem cells, embryonic cell lines, and somatic cell lines.

In certain embodiments of the invention, the pluripotent stem cells are of a livestock or poultry species. Preferably, the livestock species is porcine or bovine, preferably porcine. The pluripotent stem cells may be of the family Suidae, for example the genus Sus, such as the species S. domesticus.

In certain embodiments of the invention, the expression construct that is inserted into the second genetic safe harbour site encodes a PPAR-γ protein a linker and a CEBPα protein, preferably wherein the linker is P2A, more preferably wherein the linker comprises the sequence of SEQ ID NO: 3. Preferably, the construct comprises the sequence of SEQ ID NO: 4.

In certain embodiments of the invention, the activity of the transcriptional regulator protein is controlled by an exogenously supplied substance derivative. Preferably, the transcriptional regulator protein is selected from the group consisting of: tetracycline responsive transcriptional activator protein (rtTa), Tetracycline repressor (TetR), VgEcR synthetic receptor or a hybrid transcriptional regulator protein comprising a DNA binding domain from the yeast GAL4 protein, a truncated ligand binding domain from the human progesterone receptor and an activation domain from the human NF-kB, preferably the transcriptional regulator protein is rtTA.

In certain embodiments, the inducible promoter includes a Tet Responsive Element (TRE).

In certain embodiments, the inducible promotor is a tetON promotor.

In a second aspect the invention provides for a method for the production of adipocytes, preferably white adipocytes, comprising
a) culturing the pluripotent stem cell according to any one of the preceding claims in a proliferation medium: followed by
b) inducing adipocyte differentiation by adding the exogenous substance as described herein.

In certain embodiments, the proliferation and/or differentiation medium does not comprise or does not substantially comprise at least one compound selected from the group of insulin, dexamethasone, rosiglitazone and isobutylmethylxanthine. In certain preferred embodiments, the proliferation and/or differentiation medium does not comprise or does not substantially comprise insulin. A proliferation and/or differentiation medium which does not comprise or does not substantially comprise insulin may optionally comprise IGF-1 and/or LR3.

In certain embodiments, the differentiation phase of the method as described herein is at most 10 days, at most 9 days, 8 days, 7 days, 6 days, 5 days, 4 days, 3 days or 2 days.

In certain embodiments, the produced adipocytes are for human and non-human dietary consumption.

In a further aspect, the invention provides for a use of a pluripotent stem cell as described herein or use of the method for producing a adipocyte as described herein for tissue engineering, optionally for the production of cultured meat.

In yet a further aspect, the invention provides for a food product comprising the pluripotent stem cell as described herein or the adipocytes obtained by the method as described herein. In certain embodiments, the food product is cultured meat.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. inducible promoter.

As used herein, the term “promoter” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. In the case of the present invention, the control is effected by the transcriptional regulator protein.

Any reference to nucleotide or amino acid sequences accessible in public sequence databases herein refers to the version of the sequence entry as available on the filing date of this document.

All patent and literature references cited in the present specification are hereby incorporated by reference in their entirety.

The inventors have surprisingly found that the time period required for differentiation of pluripotent cells into mature adipocytes can dramatically be reduced by using a modified pluripotent stem cells that comprise an expression construct for expression of a PPAR-γ protein and an expression construct for expression of a CEBPα protein. As shown in the examples described herein, by using these modified pluripotent cell lines, full differentiation into mature adipocytes can be achieved in less than 10 days. Apart from significantly reducing the culturing time and the associated cost involved, the use of the herein describe pluripotent cells also provides for a more reliable and scalable production of mature adipocyte as compared to what has been previously described.

Accordingly, in a first aspect the invention relates to a pluripotent stem cell comprising:
i) an expression construct for expression of a transcriptional regulator protein inserted into a first genetic safe harbour site; 20
ii) an expression construct for expression of a PPAR-γ protein, wherein the coding sequence for the PPAR-γ protein is operably linked to an inducible promoter; and,
iii) an expression construct for expression of a CEBPα protein, wherein the coding sequence for the CEBPα protein is operably linked to an inducible promoter;
   wherein the expression constructs of ii) and iii) are inserted into at least one further genetic safe harbour site that is not the first genetic safe harbour site, and wherein the inducible promotor is regulated by the transcriptional regulator protein.

Peroxisome proliferator-activated receptor gamma (PPAR-γ) is a type II nuclear receptor functioning as a transcription factor that in humans is encoded by the PPARG gene. PPARG is mainly present in adipose tissue, colon and macrophages. Two isoforms of PPARG are detected in the human and in the mouse: PPAR-γ1 (found in nearly all tissues except muscle) and PPAR-γ2 (mostly found in adipose tissue and the intestine). In certain embodiments, the coding sequence of PPAR-γ of the invention encodes PPAR-γ2. PPARG regulates fatty acid storage and glucose metabolism. The genes activated by PPARG stimulate lipid uptake and adipogenesis by fat cells. PPARG knockout mice are devoid of adipose tissue, establishing PPARG as a master regulator of adipocyte differentiation. In certain embodiments, the coding sequence for the PPAR-γ has the sequence of SEQ ID NO: 1.

CCAAT/enhancer-binding protein alpha (CEBPα) is a protein encoded by the CEBPA gene in humans. The protein encoded by this intronless gene is a bZIP transcription factor which can bind as a homodimer to certain promoters and gene enhancers. It can also form heterodimers with the related proteins CEBP-beta and CEBP-gamma, as well as distinct transcription factors such as c-Jun. The encoded protein is a key regulator of adipogenesis (the process of forming new fat cells) and the accumulation of lipids in those cells, as well as in the metabolism of glucose and lipids in the liver. In certain embodiments, the coding sequence for the CEBPα has the sequence of SEQ ID NO: 2.

In certain embodiments, the nucleic acid molecules encoding the proteins according to the invention are codon-optimized for expression in mammalian cells. Methods of codon-optimization are known and have been described previously (e.g. WO 96/09378 for mammalian cells). A sequence is considered codon-optimized if at least one non-preferred codon as compared to a wild-type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

A transcriptional regulator protein is a protein that bind to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor). Such entities are also known as transcription factors.

The DNA sequence that a transcriptional regulator protein binds to is called a transcription factor-binding site or response element, and these are found in or near the promoter of the regulated DNA sequence.

Transcriptional activator proteins bind to a response element and promote gene expression. Such proteins are preferred in the methods of the present invention for controlling inducible cassette expression.

A genetic safe harbour (GSH) site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighboring genes and expression of the inducible cassette minimizes interference with the endogenous transcription program. More formal criteria have been proposed that assist in the determination of whether a particular locus is a GSH site in future (Papapetrou et al, 2011, Nature Biotechnology, 29 (1), 73-8. doi: 1 0. 1 038/nbt. 1 71 7.) These criteria include a site that is (i) 50 kb or more from the 5′ end of any gene, (ii) 300 kb or more from any gene related to cancer, (iii) 300 kb or more from any microRNA (miRNA), (iv) located outside a transcription unit and (v) located outside ultra-conserved regions (UCR). It may not be necessary to satisfy all of these proposed criteria, since GSH already identified do not fulfil all of the criteria. It is thought that a suitable GSH will satisfy at least 2, 3, 4 or all of these criteria.

In certain embodiments of the invention, the first and further genomic safe harbour sites are selected from any two of the hROSA26 locus, the AAVS1 locus, the CLYBL gene or the CCR5 gene. In certain embodiments the first and further genomic safe harbour sites are located on chr1: 152,360,840-152,360,859, chr1: 175,942,362-175,942,381, chr1: 231,999,396-231,999,415, chr2: 45,708,354-45, 708, 373; chr8: 68,720,172-68,720,191 of the human genome.

In certain embodiments of the invention, the first and further genomic safe harbour sites are selected from any two of the bovine safe harbour sites ROSA26, AAVS1, the CLYBL gene and the CCR5 gene.

Preferably, the genetic safe harbour sites are hROSA26 locus and the AAVS1 locus.

In certain embodiments of the invention, the expression construct for expression of PPAR-γ protein as described herein and the expression construct for expression of a CEBPα protein as described herein are both inserted into a second genetic safe harbour site that is different from the first genetic safe harbour site. In certain embodiments, the expression construct that is inserted into the second genetic safe harbour site is capable of expressing both the PPAR-γ protein and the CEBPα protein simultaneously.

As used herein, the term “pluripotent stem cells” includes embryonic stem cells, embryo-derived stem cells, induced pluripotent stem cells and somatic cells, regardless of the method by which the pluripotent stem cells are derived. Accordingly, in certain embodiments the pluripotent stem cell is selected from the group consisting of embryonic stem cells, induced pluripotent stem cells, embryonic cell lines, and somatic cell lines. In certain embodiments, the pluripotent stem cells are epiblast-derived stem cells (EpiSC). In certain embodiments, pluripotent stem cells express one or more markers selected from the group consisting of: OCT-4, Sox2, Klf4, c-MYC, Nanog, Lin28, alkaline phosphatase, SSEA-3, SSEA-4, TRA-1-60, and TRA-1-81. Exemplary pluripotent stem cells can be generated using, methods known in the art. “Induced pluripotent stem cells” (iPS cells or iPSC) can be produced by protein transduction of reprogramming factors in a somatic cell.

The pluripotent stem cell according to the invention can be from any species. Embryonic stem cells have been successfully derived in, for example, mice, multiple species of non-human primates, and humans, and embryonic stem-like cells have been generated from numerous additional species. Thus, one of skill in the art can generate embryonic stem cells and embryo-derived stem cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, gerbils, squirrel, guinea pig, goats, elephants, panda (including giant panda), pigs, raccoon, horse, zebra, marine mammals (dolphin, whales, etc.) and the like.

Similarly, iPS cells can be from any species. These iPS cells have been successfully generated using mouse and human cells. Furthermore, iPS cells have been successfully generated using embryonic, fetal, newborn, and adult tissue. Accordingly, one can readily generate iPS cells using a donor cell from any species. Thus, one can generate iPS cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like.

In certain embodiments, the pluripotent stem cell according to the invention, or for use in the invention is an animal cell. In certain embodiments the pluripotent stem cell according to the invention, or for use in the invention if from an edible animal species.

Preferably, the pluripotent stem cell according to the invention, or for use in the invention is from a livestock or poultry animal. Livestock species include but are not limited to domestic cattle, pigs, sheep, goats, lamb, camels, water buffalo and rabbits.

Preferably, the pluripotent stem cell according to the invention, or for use in the invention is a porcine or a bovine pluripotent stem cell. Most preferably, a porcine pluripotent stem cell. In certain embodiments, the stem cell according to the invention is a porcine epiblast stem cell (pEpiSCs).

Poultry species include but are not limited to domestic chicken, turkeys, ducks, geese and pigeons. In certain embodiments, the cells originate from common game species such as wild deer, gallinaceous fowl, waterfowl and hare. Preferably a pluripotent stem cell according to the invention, or for use in the invention, is not a human cell.

Transcriptional repressor proteins bind to a response element and prevent gene expression.

Transcriptional regulator proteins may be activated or deactivated by a number of mechanisms including binding of a substance, interaction with other transcription factors (e.g., homo- or hetero-dimerization) or coregulatory proteins, phosphorylation, and/or methylation. The transcriptional regulator may be controlled by activation or deactivation.

If the transcriptional regulator protein is a transcriptional activator protein, it is preferred that the transcriptional activator protein requires activation. This activation may be through any suitable means, but it is preferred that the transcriptional regulator protein is activated through the addition to the cell of an exogenous substance. The supply of an exogenous substance to the cell can be controlled, and thus the activation of the transcriptional regulator protein can be controlled. Alternatively, an exogenous substance can be supplied in order to deactivate a transcriptional regulator protein, and then supply withdrawn in order to activate the transcriptional regulator protein.

If the transcriptional regulator protein is a transcriptional repressor protein, it is preferred that the transcriptional repressor protein requires deactivation. Thus, a substance is supplied to prevent the transcriptional repressor protein repressing transcription, and thus transcription is permitted.

Any suitable transcriptional regulator protein may be used, preferably one that is activatable or deactivatable. It is preferred that an exogenous substance may be supplied to control the transcriptional regulator protein. Such transcriptional regulator proteins are also called inducible transcriptional regulator proteins.

Accordingly, in certain embodiments, the pluripotent stem cell according to the invention is controlled by an exogenously supplied substance.

In certain embodiments, the exogenously supplied substance is selected from the group consisting of peptides (such as described by Klotzsche, et al; Journal of Biological Chemistry 280.26 (2005): 24591-24599 or Schlicht et al.; Applied and environmental microbiology 72.8 (2006): 5637-5642) or the inducers described in Goeke, et al. Journal of molecular biology 416.1 (2012): 33-45; incorporated herein by reference), an aptamer (such as the RNA aptamer described in Hunsicker et al. “Chemistry & biology 16.2 (2009): 173-180; incorporated herein by reference), tetracycline, and anhydroteracyclin or a derivative thereof. Preferably, the exogenously supplied substance is doxycycline.

In certain embodiments, the transcriptional regulator protein as described herein is selected from the group consisting of tetracycline responsive transcriptional activator protein (rtTa), Tetracycline repressor (TetR), VgEcR synthetic receptor or a hybrid transcriptional regulator protein comprising a DNA binding domain from the yeast GAL4 protein, a truncated ligand binding domain from the human progesterone receptor or an activation domain from the human NF-kB.

Tetracycline-Controlled Transcriptional Activation is a method of inducible gene expression well known in the art where transcription is reversibly turned on or off in the presence of the antibiotic tetracycline or one of its derivatives (e.g. doxycycline which is more stable). In this system, the transcriptional activator protein is tetracycline-responsive transcriptional activator protein (rtTa) or a derivative thereof. The rtTA protein is able to bind to DNA at specific TetO operator sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rTA protein.

In a Tet-Off system, tetracycline or a derivative thereof binds rTA and deactivates the rTA, rendering it incapable of binding to TRE sequences, thereby preventing transcription of TRE-controlled genes. The Tet-On system is composed of two components; (1) the constitutively expressed tetracycline-responsive transcriptional activator protein (rtTa) and the rtTa sensitive inducible promoter (Tet Responsive Element, TRE). This may be bound by tetracycline or its more stable derivatives, including doxycycline (dox), resulting in activation of rtTa, allowing it to bind to TRE sequences and inducing expression of TRE-controlled genes. In preferred embodiments of the invention the transcriptional regulator protein is rtTA.

If the transcriptional regulator protein is rtTA, then the inducible promoter inserted into the at least one further GSH hat is not the first GSH site includes the tetracycline response element (TRE). Thus, in certain embodiments the inducible promoter includes a Tet Responsive Element (TRE).

In some embodiments, where the transcriptional regulator protein is rtTA and includes TRE the exogenously supplied substance is the antibiotic tetracycline or one of its derivatives.

In certain embodiments of the invention, the expression construct that is inserted into the second genetic safe harbour site is a fusion protein that encodes both the PPAR-γ protein and the CEBPα protein as described herein. In certain embodiments, expression construct that is inserted into the second genetic safe harbour site encodes a PPAR-γ protein a linker and a CEBPα protein, in preferred embodiments, the construct comprises or consists of SEQ ID NO: 4.

In certain embodiments, the linker sequence may be a cleavable linker. That is, the linker sequence may comprise a sequence of amino acids which is capable of being cleaved. For example, the linker sequence may comprise a sequence capable of acting as a substrate for an enzyme capable of cleaving peptide bonds—i.e. a cleavage site. Many such cleavage sites are known to and can be employed by the person skilled in the art of molecular biology. In some embodiments, the cleavable linker may comprise an autocleavage site. Autocleavage sites are automatically cleaved without the need for treatment with enzymes. For example the family of 2A self-cleaving peptides, or 2A peptides have been described, which includes 2A peptides P2A, E2A, F2A, and T2A. F2A is derived from foot-and-mouth disease virus; E2A is derived from equine rhinitis A virus; P2A is derived from porcine teschovirus-1 2A; T2A is derived from those a asigna virus 2A. In certain embodiments, the cleavable linker is thus selected from the group consisting of P2A, E2A, F2A, and T2A.

In some preferred embodiments the expression construct comprises a Picornavirus 2A (P2A) linker. Preferably, the expression construct comprises a linker comprising or consisting of the sequence of SEQ ID NO: 3.

In certain embodiments, the expression construct that is inserted into the second genetic safe harbour site encoding a PPAR-γ protein a linker and a CEBPα protein as described herein comprises or consists of the sequence of SEQ ID NO: 4.

In certain embodiments, the inducible promotor that is operably linked to the PPAR-γ protein is different than the inducible promotor that is linked to the CEBPα protein. In certain embodiments, the inducible promotor that is operably linked to the PPAR-γ protein is the same at the inducible promotor that is linked to the CEBPα protein. Inducible promotors are well-known in the art, examples include but are not limited to CMV, CAG, CBh, PGK, SV40, Ferritin heavy or light chains, etc.

In certain embodiments, the inducible promotor used in the present invention a tetOn promotor. Preferably a 3rd generation TetOn promotor.

### Culturing Methods

The inventors of present application have surprisingly found that the differentiation time needed to obtain adipocytes can dramatically be reduced by using the pluripotent cells as described herein. Accordingly, in a further aspect the invention relates to a method for the production of adipocytes, comprising
a) culturing the pluripotent stem cell as described herein in a proliferation medium: followed by
b) inducing adipocyte differentiation by adding an exogenous substance as described herein.

In certain embodiments, the method of the invention is an ex vivo method.

In certain embodiments, the method is for the production of mature adipocytes. Mature adipocytes are herein defined as adipocytes which show lipid accumulation and/or express detectable levels of PPARγ FABP4, PLIN1 and adiponectin.

In certain embodiments, the method of the invention relates to a method for production of white adipocytes. In certain embodiments, the proliferation and/or differentiation medium does not comprise or does not substantially comprise at least one compound selected from the group of insulin dexamethasone, rosiglitazone and isobutylmethylxanthine. In preferred embodiments, the proliferation and/or differentiation medium does not comprise or does not substantially comprise insulin.

A proliferation and/or differentiation medium which does not comprise or does not substantially comprise insulin may optionally comprise IGF-1 and/or LR3.

A proliferation and/or differentiation medium may comprise up to about 20 μg/mL insulin, for example up to about 10 μg/mL insulin, such as about 5 μg/mL insulin, for example 1 μg/ml insulin”.

The inventors have surprisingly found that use of the pluripotent cell as described herein obviates the need to culture the cells with a commitment induction step. Normally, when adipocytes are cultured several culturing phases can be distinguished. The commitment or determination phase involves the formation of preadipocytes, which have lost the potential to differentiate into other cell types. Differentiation of preadipocytes to adipocytes is promoted by a highly regulated network of transcription factors chronologically expressed to promote adipocyte morphologic and biochemical features such as insulin responsiveness, lipid transport and synthesis, and secretory capacity. The differentiation phase is also divided into four stages: growth arrest, mitotic clonal expansion, early differentiation, and terminal differentiation. Use of the pluripotent stem cells as described herein in the method as described herein allows the differentiation of the pluripotent stem cells to mature adipocytes without needing a commitment induction step and without forcing overexpression. The ability of the cells to skip this commitment induction step is particularly advantageous as it reduces the amount of compounds and small molecules that are normally required to be present in the differentiation medium. It was for example found that the cells as described herein are able to differentiate without the presence of BMP-4, Activin A and FGF2 which are normally required for satisfactory differentiation. The ability to leave out these compounds and small molecules from the differentiation medium reduces the medium costs and eases the way to regulatory acceptance. The term differentiation phase and differentiation stage are used interchangeably herein.

Accordingly, in certain embodiments the method as described herein does not comprise an additional commitment phase induction step.

The method as described herein reduces the differentiation time of the pluripotent cells as described herein to mature adipocytes dramatically. In certain embodiments, the time to produce mature adipocytes using the method as claimed is at most 10 days, 9 days, 8 days, 7 days, 6 days, 5 days, 4 days, 3 days or 2 days. Using the pluripotent cells as described in the method as described, the inventors have observed a conversion rate of at least 95% by day 4 of culture, meaning that at least 95% of the cells are mature after 4 days of culture. Accordingly, in certain embodiments the time to produce at least 95% mature adipocytes is at most 4 days.

In a further aspect, the invention provides for adipocytes, preferably mature adipocytes, as obtained by the method as described herein.

Culturing the cells as described herein can be performed under so called 2D culturing conditions, which is considered the conventional approach to culturing cells. However, the method as described can also easily be adapted to allow culturing under 3D conditions as shown in the examples below.

3D cell culture is an artificially-created environment which enables cells to grow or interact with their surroundings in three dimensions. In such culture, cells typically form 3D colonies, which may be referred to as “spheroids”. The 3D culture approach may more accurately model the cells' in vivo growth and behaviour. The skilled person is readily able to carry out 3D cell culture, for example by taking advantage of any of a number of commercially-available culturing tools. For example, the 3D culture may be carried out using scaffold or scaffold-free techniques. Scaffold-based techniques make use of supports such as solid scaffolds and hydrogels to enable the cells to form a 3D culture. Such scaffolds may aim to mimic the natural extracellular matrix (ECM), which is present in vivo. Scaffold-free techniques dispense with the use of the scaffold on which to grow the cells. Instead, 3D spheroids may be established through the use of, for example, low-adhesion plates, hanging-drop plates, micro-patterned surfaces, rotating bioreactors, magnetic levitation and magnetic 3D bioprinting.

Cells that have been transduced with lentiviral vectors are not considered food safe or not safe for human and non-human dietary consumption. The pluripotent cells as described herein of the method as described herein obviates the need to use lentivirally transduced cells. Accordingly, in certain embodiments, the adipocytes that are produced according to the method as disclosed herein are for human and non-human dietary consumption. In certain embodiments, the produced adipocytes can be used in the production of cultured meat for human consumption.

In a further aspect, the invention provides for a use of a pluripotent stem cell as described herein or the adipocytes obtained by the method as described herein for tissue engineering. In certain embodiments the method as described herein is for ex vivo or in vivo tissue engineering.

In certain aspect, the use is for the production of cultured meat. That is to say, the invention provides for a use of a pluripotent stem cell as described herein or the adipocytes obtained by the method as described herein for the production of cultured meat.

In yet a further aspect, the invention provides for a food product (also referred to as “foodstuff”) comprising the pluripotent stem cells as described herein or the adipocytes produced and/or obtained by the method as described. In certain embodiments, the food product is or further comprises an edible composition for human or non-human consumption. The edible composition for human or non-human consumption for example comprises at least one of myocytes, mature muscle cells, minerals, synthetic substances, flavoring substances (such as for examples herbs and spices), plant based proteins or proteins from microbial origin such as yeast proteins. Plant based proteins and yeast proteins suitable for the use in food products are known to the skilled person in the art. In certain embodiments, the food product is cultured meat or a cultured meat product.

In yet a further aspect, the invention provides for a method of producing a food product, the method comprising combining the pluripotent stem cells as described herein or the produced and/or obtained adipocytes with an edible composition for human consumption or non-human consumption as described herein. In certain embodiments, the food product is cultured meat.

### Description of the Sequences

TABLE 1
Sequences

| SEQ ID NO: | Name |
|---|---|
| 1 | DNA sequence encoding the PPAR-γ protein |
| 2 | DNA sequence encoding the CEBPα protein |
| 3 | GSG-P2A_linker |
| 4 | PPARG-P2A-CEBPA - construct |
| 5 | pMI014_PPARG-P2A-CEBPA_opti-ox-aavs1 |

### DESCRIPTION OF THE FIGURES

FIG. 1: Comparison between EpiSCs-PPARγ and EpiSCs-PPARγ-CEBPα 2D differentiation. Undifferentiated (day 0) and differentiated (days 2, 4, 6 and 8) EpiSCs-PPARγ and EpiSCs CEBPα-PPARγ. Bright-field microscopy, 10×.

FIG. 2: mRNA relative gene expression for porcine PPARγ and CEBPα Opti-Ox cells. A) PPARγ, B) CEBPα, C) endogenous PPARγ D) endogenous CEBPα E) adiponectin, F) LPL, G) Perilipin-1, H) FABP4, i) CD36 and J) CEBPβ, K) ZBTB16 and L) Oct 4. EpiSCs-PPARγ and EpiSCs CEBPα-PPARγ differentiated in 2D for 8 days.

FIG. 3: Total intracellular triglycerides quantification for undifferentiated EpiSCs-PPARγ and EpiSCs-PPARγ-CEBPα and after 2, 4, 6 and 8 days of differentiation.

FIG. 4: Comparison between EpiSCs-PPARγ and EpiSCs-PPARγ-CEBPα 2D differentiation. Undifferentiated (day 0) and differentiated (days 7, 14 and 20). Fluorescent microscopy, 10×. Stained with Oil Red O (red, neutral lipids) and DAPI (blue, nuclei).

FIG. 5: Comparison between EpiSCs-PPARγ and EpiSCs-PPARγ-CEBPα 3D differentiation. EpiSCs-PPARγ and EpiSCs PPARγ-CEBPα differentiated in suspension (3D, day 6) stained with Oil Red O (red, neutral lipids) and DAPI (blue, nuclei). Fluorescent microscopy, 10×.

FIG. 6: EpiSCs PPARγ-CEBPα differentiated (days 4, 12 and 20) under protocol D (Mesodermal step+Maturation step) or protocol F (only maturation step). A) Bright-field microscopy, 40×. B) Quantification (absorbance) of the intracellular Oil Red O staining extracted from EpiSCs CEBPα-PPARγ differentiated for 20 days under protocol D or protocol F conditions.

FIG. 7: EpiSCs PPARγ-CEBPα differentiated (days 2, 7 and 13) under protocol D (Mesodermal step+Maturation step) with maturation media supplemented with (1) KSR, insulin and dexamethasone (2) KSR and dexamethasone or (3) KSR and insulin; or under protocol F (only maturation step) with maturation media supplemented according to conditions (1), (2) and (3). Bright-field microscopy, 20×.

FIG. 8: EpiSCs PPARγ-CEBPα differentiated in suspension (3D, day 13) under protocol D (Mesodermal step+Maturation step) with maturation media supplemented with (1) KSR, insulin and dexamethasone (2) KSR and dexamethasone or (3) KSR and insulin; or under protocol F (only maturation step) with maturation media supplemented according to conditions (1), (2) and (3). Aggregates stained with Oil Red O (red, neutral lipids) and DAPI (blue, nuclei). Fluorescent microscopy, 10×.

FIG. 9: EpiSCs PPARγ-CEBPα differentiated in suspension (3D, day 7) in a bioreactor under a fed batch protocol that is similar to protocol D (Mesodermal step+Maturation step) with media supplemented with insulin and dexamethasone. Aggregates were collected on day 0, 3, 5 and 7 of differentiation and total intracellular triglycerides quantified.

FIG. 10: EpiSCs PPARγ-CEBPα differentiated (day 7) under protocol F (only maturation step) with maturation media supplemented with insulin at (A) 20 μg/mL, (B) 10 μg/mL, (C) 5 μg/mL or (D) without insulin. Maturation media was also supplemented with (E) 0.05 μg/mL IGF-1 or (F) 0.05 μg/mL IGF-1/LR3 in the absent of insulin. Bright-field microscopy, 20×.

### EXAMPLES

The present invention is further described by the following examples which should not be construed as limiting the scope of the invention.

### Materials and Methods

Porcine Epiblast-Derived Stem Cells (pEpISCs) Differentiation to Adipocytes.

Undifferentiated pEpISCs (Opti-Ox PPARγ and Opti-Ox CEBPα-PPARγ) were grown in N2B27 proliferation media (50% DMEM Ham's F-12 (L0093-500, Biowest), 50% Neurobasal media (21103049, Thermofisher), B27 supplement (17504044, Thermofisher), N2 supplement (17502001, Thermofisher), glutamax (35050061, Thermofisher), 10 mM 2-Mercaptoethanol (31350010, Thermofisher), 0.02 μg/mL Activin A (QK001, Q-kine), 0.10 μg/mL FGF2 (QK002, Q-kine), 0.625 μg/mL XAV939 (X3004, Sigma Aldrich) on hESC-qualified geltrex (A1413301, Thermo Scientific)-coated plates. For 2D adipocyte differentiation, single cells were obtained after gentle cell dissociation reagent (07174, STEMCELL technologies) treatment and cell number and viability were assessed. Single pEpISCs cells were seeded in the corresponding cell culture plates at a density of 50000-150000 cells/cm2. After an overnight incubation in proliferation media with 10 μm Rock Inhibitor (Y-27632 (HBF2297, HelloBio), cells were cultivated in StemPro-34 SFM media (10639011, Thermo Scientific) supplemented with 25 ng/ml Activin A (120-14E, PeproTech), 10 ng/mL BMP4 (120-05ET, PeproTech), 4 ng/ml FGF2 (Qk002, Qkine) and 50 mg/mL Ascorbic acid (A8960, Sigma Aldrich) for 48 hours to enhance stem cells commitment to adipocyte lineage. On day 2, media was replaced by adipocyte maturation media composed by DMEM Ham's F-12 (L0093-500, Biowest) containing 15% Knockout Serum Replacement (10828-028, Thermofisher), 1 μg/mL insulin (12585014, Thermofisher) and 1 μM Dexamethasone (D1756, Sigma Adrich). pEpISCs were differentiated for the specified amount of days. Media was refreshed every two days. Doxycycline (1 μg/mL, D9891, Sigma Aldrich) was added to the differentiation media to activate the Opti-OX system in these cells.

For spheroids or aggregates differentiation experiments, undifferentiated single-pEpISCs (Opti-Ox PPARγ and Opti-Ox CEBPα-PPARγ) were seeded at 3 million cells/mL in 250 ml shaker flasks containing 25 mL of proliferation media (containing 50% DMEM Ham's F-12 (L0093-500, Biowest), 50% Neurobasal media (21103049, Thermofisher), B27 supplement (17504044, Thermofisher), N2 supplement (17502001, Thermofisher), glutamax (35050061, Thermofisher), 10 mM 2-Mercaptoethanol (31350010, Thermofisher), 0.02 μg/mL Activin A (QK001, Q-kine), 0.10 μg/mL FGF2 (QK002, Q-kine), 0.625 μg/mL XAV939 (X3004, Sigma Aldrich), 2× % Knockout Serum Replacement (10828-028, Thermofisher) KSR and 10 ng/mLX FGF2). Next day, once small spheroids were formed, media was replaced by supplemented StemPro-34 SFM. In the same way, maturation media was added at day 2 of differentiation and was refreshed every two days until the end of the experiment.

### Oil Red O Staining

Undifferentiated and differentiated pEpISCs Opti-Ox PPARγ and pEpISCs Opti-Ox CEBPα-PPARγ were stained with Oil Red O as previously described (O0625, Sigma). Briefly, cells in culture or in aggregates were washed with phosphate buffered saline (PBS) and fixed with 4 paraformaldehyde (PFA) for 40 minutes at room temperature. After being washed 3 times with PBS and permeabilized with 60% isopropanol, cells were stained with Oil Red O (0.5% in isopropanol) diluted to 40% with water, for 30 minutes at room temperature. Thereafter, pEpISCs were washed several times with deionized water until only lipid droplets were properly stained with the dye.

Undifferentiated and differentiated cells were photographed under a inverted bright fieldlight microscope (Oxion Inverso, Euromex) or under fluorescent microcopya (EVOS M7000, Thermo Fisher Scientific). Triglyceride content of pEpISCs Opti-Ox PPARγ and pEpISCs Opti-Ox CEBPα-PPARγ was estimated at different check points during adipogenesis by extracting Oil Red O dye from the cells with 100% isopropanol and measuring spectrophotometrically the resulting solution at 540 mM (Glomax Discover, Promega). Oil Red O data were adjusted by an estimation of the number of cells spectrophotometrically measured by HOESCH 33342 staining (H1399, Thermo Fisher Scientific), according to the manufacturer's instructions.

pEpISCs Opti-Ox PPARγ and pEpISCs Opti-Ox CEBPα-PPARγ differentiated in aggregates or spheroids were stained following the same protocol previously described. Two different approaches were followed for imaging acquisition. For a quick check on adipocyte quality, stained aggregates were transferred to a slide. After removing excess buffer, the slides were coverslipped using hard set mounting medium (P36984, Thermo Fisher Scientific). In this process the spheroid aggregates are flattened, resulting in an overview of lipid presence and an estimate of its distribution. For a more accurate analysis, unstained and fixed cell aggregates were cryopreserved in sucrose solution. The frozen samples were then sectioned at 20 μm with a cryostat (CM 1950, Leica). The cryosections were stained using the Oil Red O staining protocol previously described. Measurements included total cell number, total lipid number per cell, lipid size and shape, total intensity (within lipid objects).

### Triglyceride Content

Intracellular triglyceride content was quantified from undifferentiated and differentiated cultured/aggregates pEpISCs Opti-Ox PPARγ and pEpISCs Opti-Ox CEBPα-PPARγ using Triglyceride Quantification Colorimetric/Fluorometric Kit (MAK266-1KT, Sigma Aldrich) according to manufacturer's instructions. Briefly, total lipids were extracted with 5% Nonidet P40 Substitute (11754599001, Sigma Aldrich) solution in deionized water under heating. By adding lipase, glycerol was released from triglycerides and subsequently reacted to generate colour, which can be measured spectrophotometrically at 570 nm (Glomax Discover, Promega). TG concentrations were calculated based upon a standard curve made from TG standards and normalized to total cellular protein content.

### Real-Time Quantitative PCR Analysis (RT-qPCR)

Total RNA from undifferentiated and differentiated pEpISCs Opti-Ox PPARγ and pEpISCs Opti-Ox CEBPα-PPARγ was extracted using Reliaprep Cell Miniprep System (Z6012, Promega) according to the manufacturer's instructions. RNA concentration and quality was determined with a Microvolume Spectrophotometer DS-11 (DeNovix). Five hundred nanograms of purified total RNA from every sample were first treated with DNase I to remove possible genomic DNA contamination and were subsequently reverse transcribed into cDNA using iScript gDNA Clear cDNA Synthesis Kit (1725035BUN, BioRad). Specific primers for porcine pluripotency and mature adipocyte markers were designed to perform real-time quantitative PCR analysis (table I). At least three samples from two independent experiments were amplified in triplicates in an (thermocycler) system using PowerTrack SYBR Green Master Mix (A46112, Thermofisher) according to manufacturer's guidelines. RT-qPCR conditions were 95 °C for 30 seconds, followed by 40 cycles of 15 s at 95 °C and 1 min at 60 °C. The YWHAZ gene was used as a housekeeping gene to normalize the target gene expression levels. Relative gene quantification was calculated by the 2-ΔΔCt method.

### Results

### Example 1: Development of an Inducible Transgene Overexpression Method by Dual GSH Targeting in Animal Cells

To explore the potential of OPTi-OX for forward programming of porcine (pPSCs), we generated PPARG OPTi-OX pPSCs. We sequentially targeted the rtTA cassette into the porcine ROSA26 GSH under control of a CAG promoter and a PPARG transgene into the porcine AAVS1 GSH under control of a doxycycline-inducible element and observed robust and homogeneous inducible transgene expression. Induction of PPARG expression following dox treatment resulted in cells accumulating lipids. These findings demonstrated that PPARG overexpression alone was sufficient to drive adipogenesis in pPSCs. However, pEpiSCs PPARg fat accumulation and cell morphology were far from being comparable to porcine mature adipocytes and some specific late adipogenesis markers, such as PLIN1 or Adiponectin, were not expressed even after 21 days of differentiation.

After conducting a systematic screen for adipogenic factors by modulating key signaling cascades that are implicated in adipogenesis, PPARg and CEBPa were selected for used in a combined cellular reprogramming strategy. We designed a knock-in that has PPARg, a P2A “self-cleaving” peptide linker and CEBPa all in one open reading frame to simultaneously express PPARg and CEBPa by doxycycline induction. 2A peptide linkers are well-characterized short peptide linkers of 18-22 amino acids that result in two separate gene products expressed from a single open reading frame due to ribosome skipping during translation. Stable knock-ins in the AAVS1 GSH were selected by incorporation of a puromycin-resistance cassette and selected with puromycin addition to the cell culture medium. Following selection, single pEpiSC cells were plated and clonal cell lines were isolated for outgrowth and analysis. Incorporation of doxycycline-inducible PPARg-P2A-CEBPa was subsequently confirmed using PCR genomic analysis, Sanger sequencing and RT-qPCR. Using the dual GSH-targeting approach, we selected clonal lines that carried two copies of each of the transgenes, and observed that homozygous targeting of both elements allowed inducible overexpression (data not shown). Importantly, the dual GSH-targeting approach did not affect SC self-renewal or differentiation as determined by RT-qPCR (data not shown).

### Example 2: EpiSCs-PPARγ-CEBPα 2D Short-Term Differentiation into Adipocytes

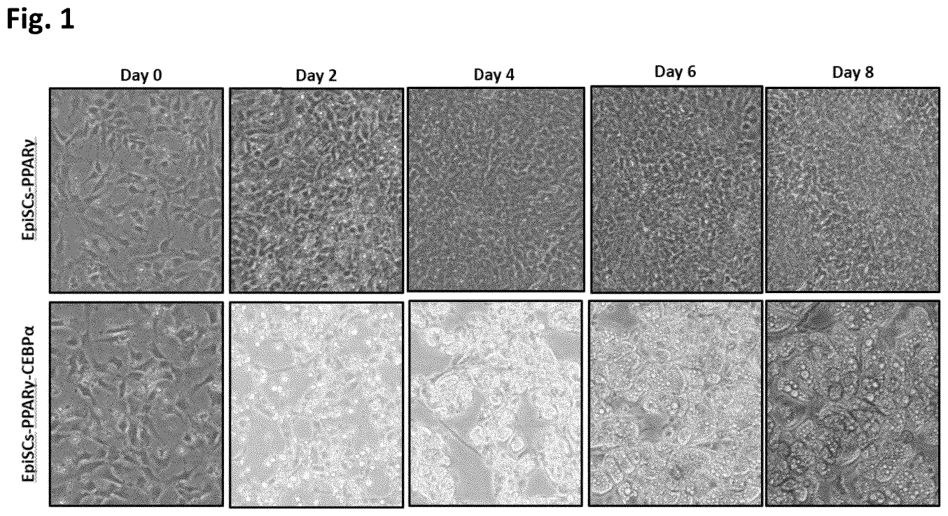

pEpISCs Opti-Ox PPARγ and pEpISCs Opti-Ox PPARγ-CEBPα were differentiated with doxycycline for up to 8 days. Pictures were taken from the undifferentiated cells (day 0) and during differentiation (at days 2, 4, 6 and 8) using bright-field microscopy. As can be seen in FIG. 1, pEpISCs comprising Opti-Ox-PPARγ-CEBPα created bigger lipid droplets during differentiation than pEpiSC PPARγ indicating better cell maturation and more lipid accumulation. This difference was already visible at day 4 after the start of differentiation indicating a much more rapid differentiation for pEpISCs comprising Opti-Ox PPARγ-CEBPα.

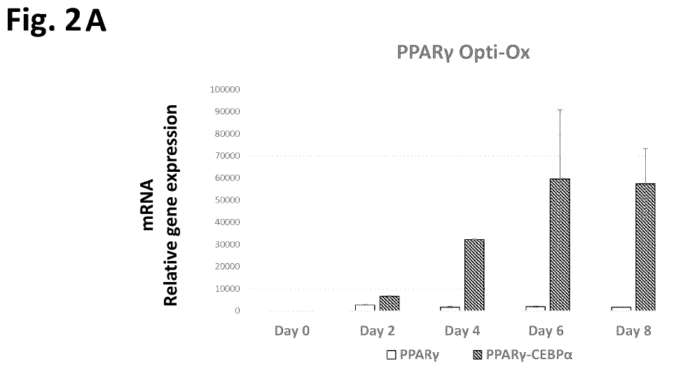

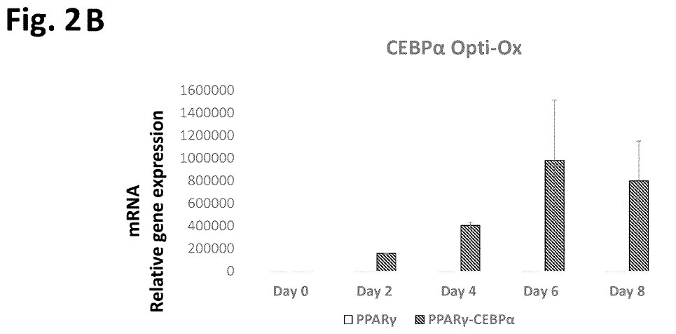

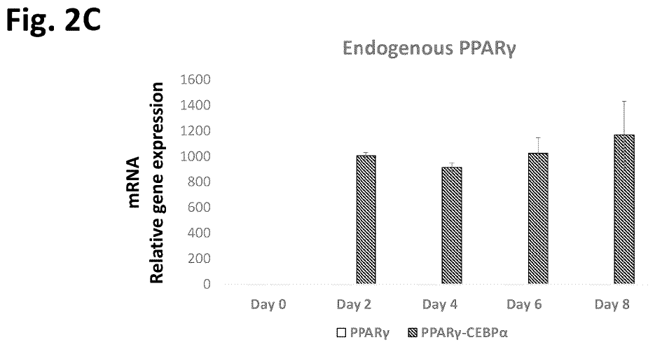

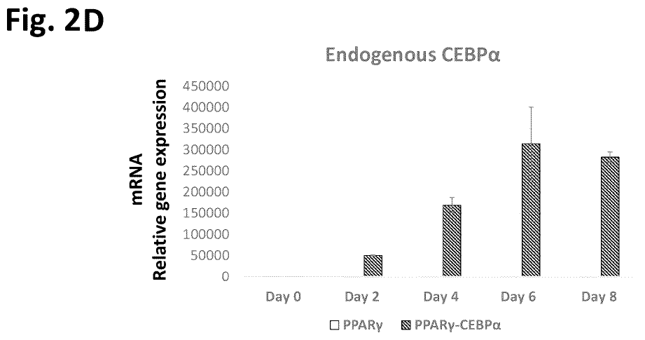

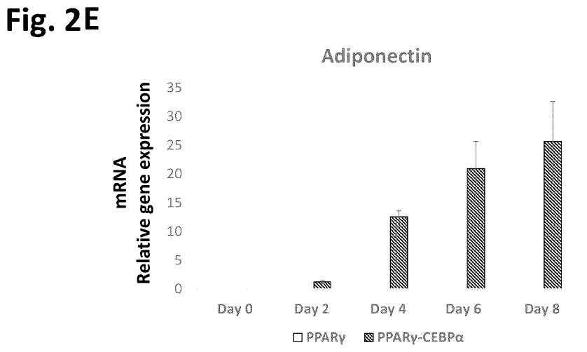

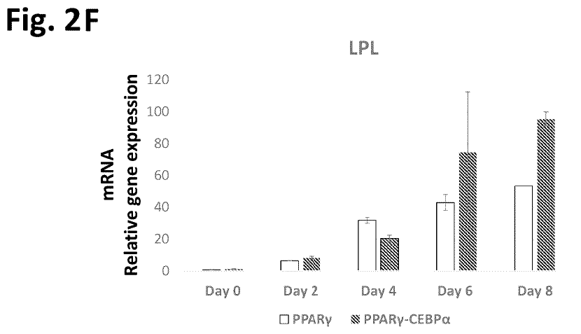

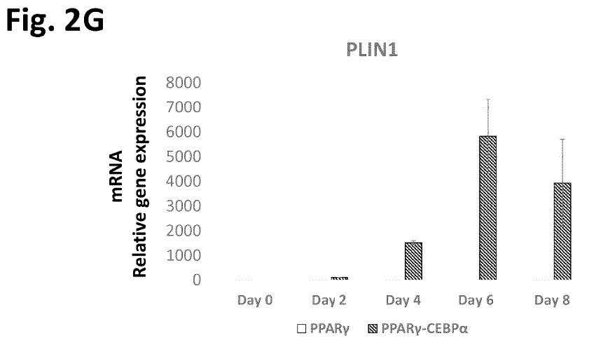

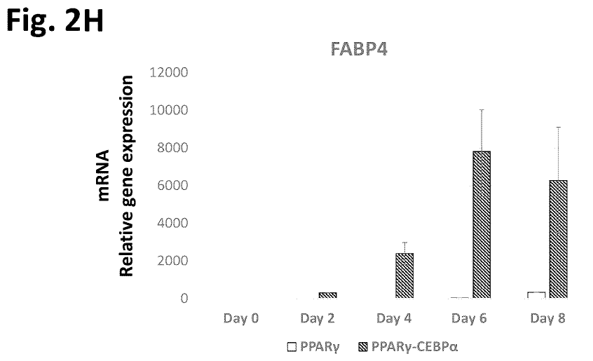

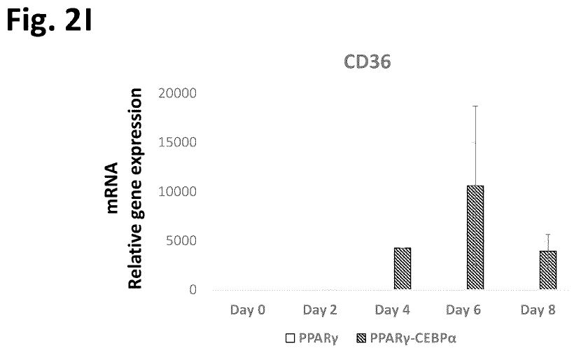

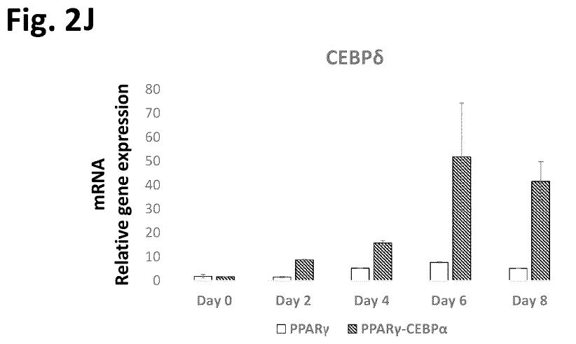

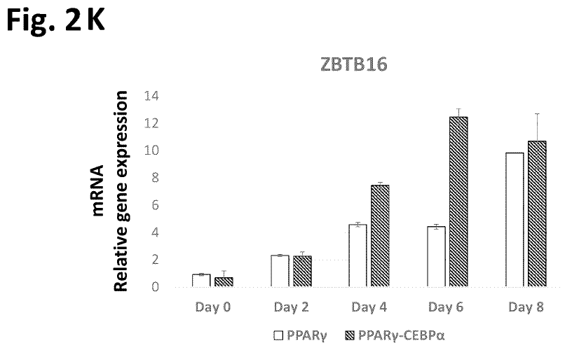

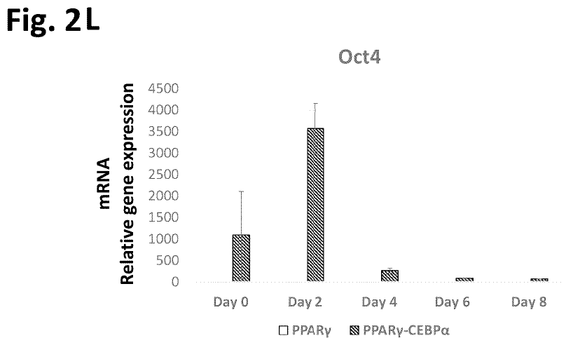

We used RT-qPCR to assess mRNA relative gene expression for porcine endogenous PPARγ, CEBPα, adiponectin, LPL, Perilipin-1, FABP4, CD36 and CEBPδ, results are shown in FIG. 2. As expected, pEpiSC PPARγ-CEBPα cells express Opti-Ox CEBPα and Opti-Ox PPARγ, already after 2 days of differentiation, which confirms that the Opti-Ox system is capable of stable transgene expression and confirms the useability of the cell model. pEpiSC PPARγ-CEBPα cells had higher expression of several adipocyte markers (LPL and CD36, Perilipin 1 (indicating lipid accumulation), CEBPδ and ZBTB16 (late adipogenesis markers), Adiponectin and FABP4 (markers of terminal cell differentiation). Adiponectin and Perilipin 1 were not expressed in the pEpiSC PPARγ. Indicating that pEpiSC PPARγ-CEBPα have a higher differentiation potential. Additionally, we observed that Oct4 expression is induced in pEpiSC PPARγ-CEBPα at day 2. This effect is not observed in pEpiSC PPARγ. Oct4 is known to be an important modulator for the commitment of the cells to the adipocyte lineage indicating that the pEpiSC PPARγ-CEBPα do not need a commitment step during differentiation which allows for quicker differentiation.

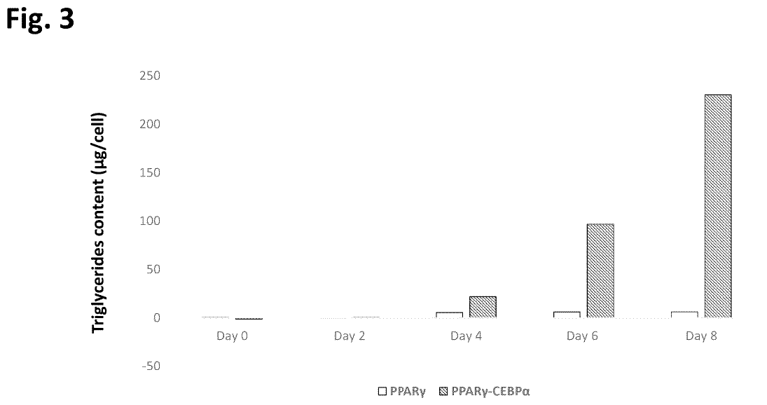

Next, we quantified the total triglyceride content from the undifferentiated and differentiated cells (FIG. 3) using Oil Red O staining. Oil Red O is a fat-soluble dye that stains neutral triglycerides and lipids. The results show that pEpiSC PPARγ-CEBPα had a significantly higher triglyceride content indicating that their differentiation is faster and more efficient when compared to pEpiSC PPARγ.

### Example 3: EpiSCs-PPARγ-CEBPα 2D Long-Term Differentiation into Adipocytes

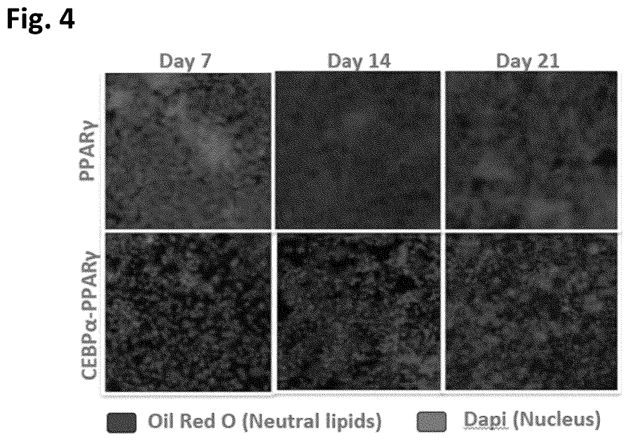

We repeated the experiment from example 2 but now differentiated the pEpISCs Opti-Ox PPARγ and pEpISCs Opti-Ox PPARγ-CEBPα cells for up to 20 days and collected the differentiated cells at Day 7, 14 and 21 for Oil Red O Staining (FIG. 4). pEpiSC CEBPα-PPARγ cells accumulates bigger lipid droplets than pEpiSC PPARγ during adipogenesis. The difference is already detectable at day 7, with the pEpISCs Opti-Ox PPARγ-CEBPα having a well-defined adipocyte-like shape and homogenous lipid accumulation. Additionally, pEpiSC PPARγ-CEBPα cells do not need to reach confluency before starting differentiation (data not shown) indicating that pEpiSC PPARγ-CEBPα are less dependent of external pro-adipogenic factors (ECM, secreted factors).

### Example 4: EpiSCs-PPARγ-CEBPα Skip the Commitment Step During Differentiation

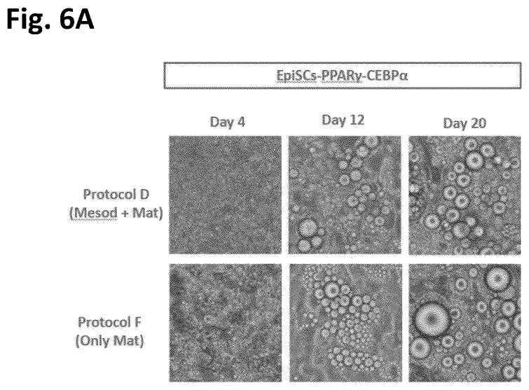

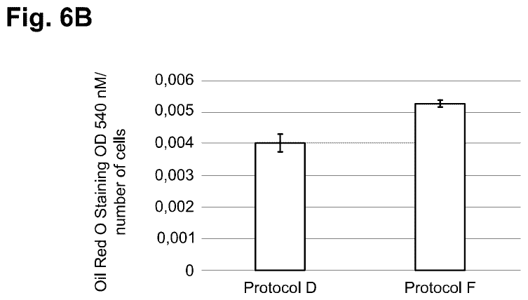

To confirm that a commitment differentiation step was not required EpiSCs-PPARγ-CEBPα were differentiated in 2D using two differentiation protocols. Protocol D (including commitment+maturation step) and protocol F (only maturation step). Cells were collected during differentiation at days 4, 12 and 20 and analyzed under bright-field microscopy (FIG. 6A). Neutral lipid content was estimated by Oil Red O adjusted by the number of cells measured with the HOESCH method. Both dyes were spectrophotometrically quantified (FIG. 6B).

While the commitment step was necessary to improve the differentiation capacity of the pEpiSC PPARγ (data not shown), EpiSCs-PPARγ-CEBPα adipogenesis performance was comparable between protocol D and F (FIG. 6A). As a matter of fact, neutral lipid accumulation per cell was higher in the EpiSCs-PPARγ-CEBPα differentiated in the absence of the mesodermal step (FIG. 6B), which makes this model more promising in terms of cost-reduction (process and media) and because several small molecules can be left out for regulatory acceptance of cultured meat.

### Example 5: EpiSCs-PPARγ-CEBPα 2D Differentiation: Differentiation Using Protocol D and Protocol F with KSR+Insulin+Dexamethasone, with KSR+Dexamethasone (No Extra Insulin) and with KSR+Insulin (No Dexamethasone)

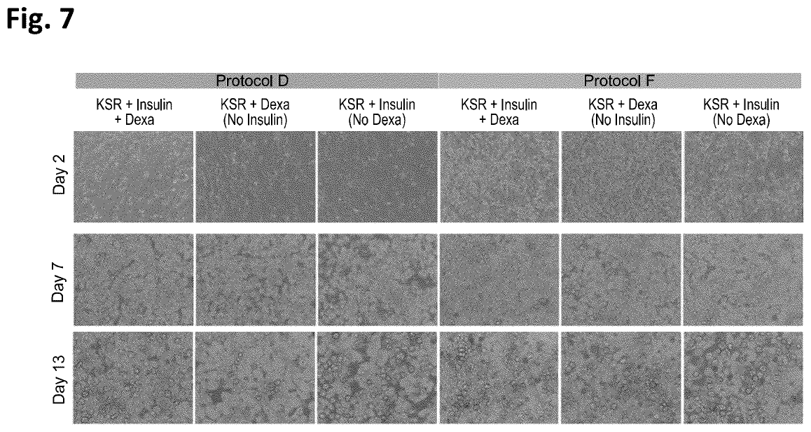

To explore if EpiSCs-PPARγ-CEBPα need additional insulin and dexamethasone in the maturation step of differentiation, EpiSCs-PPARγ-CEBPα were differentiated in 2D using differentiation protocols D and F in the presence or absence of additional insulin and/or dexamethasone. Cells were collected during differentiation at days 2, 7 and 13 and analyzed under bright-field microscopy (FIG. 7A). Adipogenesis performance was comparable between both Protocol D and F with insulin and dexamethasone and these protocols without insulin and/or dexamethasone. Being able to leave out insulin and dexamethasone makes this model more promising in terms cost-reduction (process and media) and because several small molecules can be left out for regulatory acceptance of cultivated meat.

### Example 6: 3D Culture of EpiSCs-PPARγ-CEBPα

In suspension culture of cells in 3D is a necessary step to scale up to large volumes and generate the amounts of adipocytes needed for a cultivated meat product at cost competitive prices (for example in shakers and/or bioreactors). Although in suspension culture of cells in 3D allows the cells to grow in an environment that more closely mimics the physiological in vivo environment of the cells the transition from 2D to 3D culture needs to be properly performed and differentiation data obtained from 2D experiments need to be validated in 3D.

For this reason, EpiSCs-PPARγ-CEBPα were adapted to 3D suspension cell growth and grown as aggregates; EpiSCs-PPARγ-CEBPα expanded in an adherent 6-well cell plate were single celled with Accumax (00-4666-56, ThermoFisher Scientific) according to the manufacturer's instructions and transferred to a 150 mL shaker flask in 12.5 mL media and a RHO/ROCK pathway inhibitor and thereafter expanded for at least 3 cycles. Media during expansion in 3D was refreshed daily. 3D adapted EpiSCs-PPARγ-CEBPα were subsequently used in shaker and bioreactor experiments.

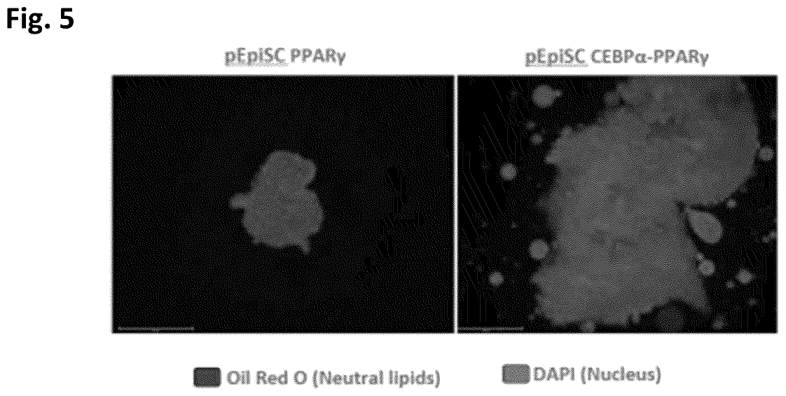

To explore adipogenesis performance in 3D, 3 million cells/mL were inoculated in a 250 mL shaker flask containing 25 ml of media and EpiSCs-PPARγ-CEBPα were differentiated using differentiation protocol D in the presence of additional insulin and dexamethasone. Media was changed every second day. Cells were collected during differentiation at day 6, stained with Oil Red O and DAPI and visualized by fluorescent microcopy using the pancake methodology previously described (FIG. 5) mRNA relative gene expression for porcine PPARγ and CEBPα Opti-Ox-derived mRNA and porcine endogenous PPARγ, CEBPα, adiponectin, LPL, Perilipin-1, FABP4, CD36 and CEBPβ was analyzed as previously described herein. The relative gene expression did not differ significantly from the relative gene expression observed in cells grown in 2D culture (data not shown) confirming that the adipocytes were suitably adapted to growth and differentiation in 3D culture.

### Example 7: EpiSCs-PPARγ-CEBPα 3D Differentiation in Suspension: Protocol Optimization: Differentiation Using Protocol D and Protocol F with KSR+Insulin+Dexamethasone, with KSR+Dexamethasone (No Extra Insulin) and with KSR+Insulin (No Dexamethasone)

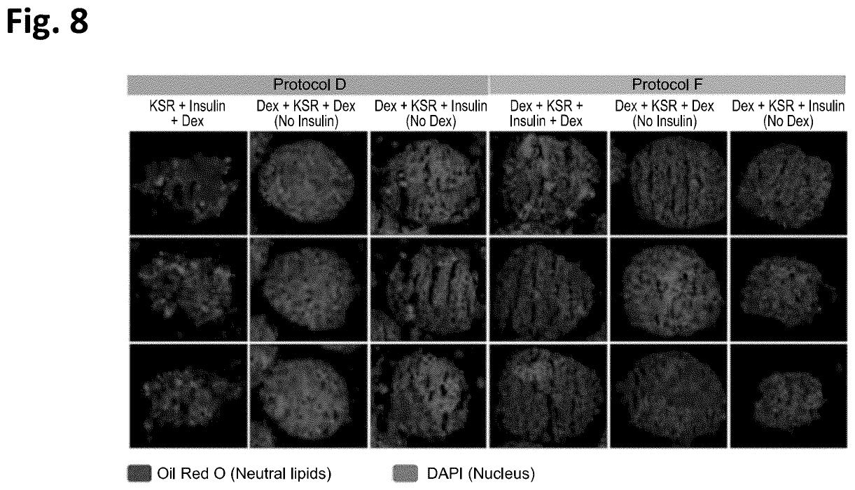

To confirm that in 3D also additional insulin and dexamethasone can be left out, EpiSCs-PPARγ-CEBPα were differentiated in shakers and the setup of example 5 was repeated in 3D. pEpiSC PPARγ-CEBPα differentiated both, under the protocol D (mesodermal step+maturation step) and the protocol F (only maturation step) in 3D in suspension as well (FIG. 8). Intracellular triglycerides were also detectable in the center of the aggregate which points to a sufficient nutrient and factor perfusion inside of the 3D structure (including size aggregate). Thus pEpiSC PPARγ-CEBPα are also capable of differentiation into adipocytes with no additional insulin and with no dexamethasone added in the maturation media, again confirming this model is more promising in terms cost-reduction (process and media) and because several small molecules can be left out for regulatory acceptance of cultivated meat.

### Example 8: EpiSCs-PPARγ-CEBPα 3D Differentiation in Suspension in Bioreactor

To explore if pEpiSC PPARγ-CEBPα can be grown and differentiated in a bioreactor, a fed batch procedure was designed using an Ambr250 modular bioreactor system (Sartorius), where 6×10⁶/mL undifferentiated cells were inoculated in 120 mL DMEMF/12 medium (21041-025, Gibco) supplemented with KSR, BMP4, FGF2, Activin A, Glutamax and Ascorbic acid in the same concentrations described for previous experiments. Glucose concentration was maintained above 7 mM, temperature was kept at physiological levels, agitation speed was sufficient to assure aggregate size do not exceed the limits for nutrient perfusion, 7.5% Sodium bicarbonate was added on demand to maintain pH at 7.4±0.5 and dissolved oxygen set-point was established between 30-70%. Additional insulin and doxycycline were added on days 0, 2, 4 and 6 while dexamethasone was only added once on day 2. Aggregates were collected on day 0, 3, 5 and 7 of differentiation for further analysis. Media components concentrations were monitored every 24 hours to identify possible limitations (data not shown). Samples for neutral lipid quantification and gene expression analysis were obtained on days 0, 3, 5 and 7. Aggregate's size were measured on day 0 and 6.

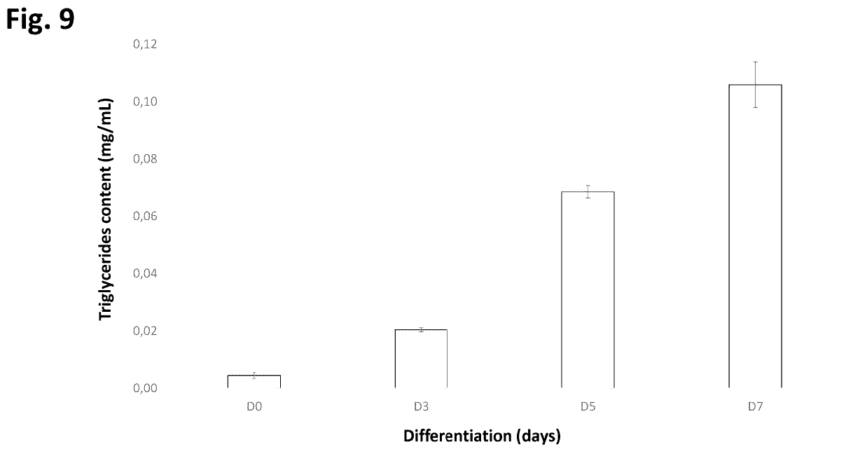

Intracellular triglycerides were already detected after 3 days of differentiation in pEpiSC PPARγ-CEBPα, while lipid accumulation was constantly increasing until the end of the experiment at day 7 of differentiation (FIG. 9). These results confirm that that pEpiSC PPARγ-CEBPα can differentiate into adipocytes in a bioreactor, which highlight the potential of this model to be used in scaling-up processes.

### Example 9: EpiSCs-PPARγ-CEBPα Differentiation in the Absence of Insulin

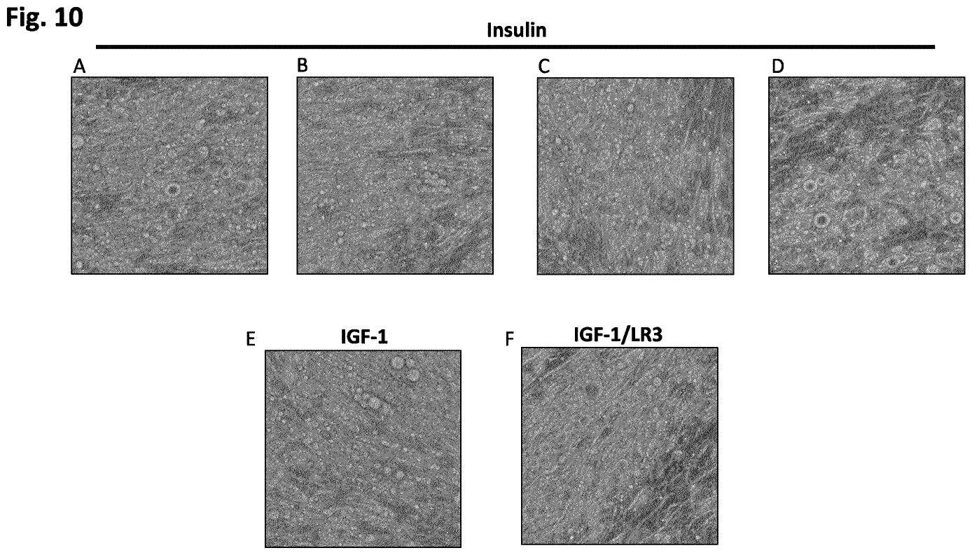

PPARγ-CEBPα pEpISCs were seeded at 125000 cells/cm² in 6-well plates pre-coated with Geltrex, in proliferation media+Fasudil at 3.5 μM. Cells were homogeneously distributed in the wells and incubated overnight at 38.5 degrees, 5% CO₂ to ensure their attachment. Adipocyte differentiation was induced the next day by refreshing the media with maturation media (Protocol F) supplemented with 0.5 μg/mL doxycycline and containing 20 μg/mL insulin (control, condition FIG. 10A), 10 μg/mL insulin (condition FIG. 10B), 5 μg/mL insulin (condition FIG. 10C) or in the absence of insulin (condition FIG. 10D). Free-insulin maturation media was also supplemented with 0.05 μg/mL IGF-1 (Condition FIG. 10E) and 0.05 μg/mL IGF-1/LR3 (condition FIG. 10F). Maturation media was replaced every two days and cells were incubated at 38.5 degrees, 5% CO₂ during differentiation. Bright-field images were taken on day 8 of differentiation. These results confirm that insulin is not required for efficient differentiation.

Statements (features) and embodiments of the methods and compositions as disclosed herein are set out below. Each of the statements and embodiments as disclosed by the invention so defined may be combined with any other statement and/or embodiment unless clearly indicated to the contrary. In particular, any feature indicated as being preferred or advantageous may be combined with any other feature or features indicated as being preferred or advantageous.

### Embodiments

The present invention provides at least the following numbered statements/embodiments:
1. A pluripotent stem cell comprising:
i) an expression construct for expression of a transcriptional regulator protein inserted into a first genetic safe harbour site;
ii) an expression construct for expression of a PPAR-γ protein, wherein the coding sequence for the PPAR-γ protein is operably linked to an inducible promoter; and,
iii) an expression construct for expression of a CEBPα protein, wherein the coding sequence for the CEBPα protein is operably linked to an inducible promoter;
wherein the expression constructs of ii) and iii) are inserted into at least one further genetic safe harbour site that is not the first genetic safe harbour site, and wherein the inducible promotor is regulated by the transcriptional regulator protein.
2. A pluripotent stem cell according to embodiment 1, wherein the expression constructs of ii) and iii) are both inserted into a second genetic safe harbour site that is different from the first genetic safe harbour site.
3. The pluripotent stem cell according to embodiment 1 or 2, wherein the cell is selected from the group consisting of embryonic stem cells, induced pluripotent stem cells, embryonic cell lines, and somatic cell lines.
4. The pluripotent stem cell according to any one of embodiments 1-3, wherein the pluripotent stem cells are of a livestock or poultry species.
5. The pluripotent stem cell according to embodiment 4, wherein the livestock species is porcine or bovine, preferably porcine.
6. The pluripotent stem cell according to any one of embodiments 2-5, wherein the expression construct that is inserted into the second genetic safe harbour site encodes a PPAR-γ protein a linker and a CEBPα protein, preferably wherein the linker is P2A, more preferably wherein the linker comprises the sequence of SEQ ID NO: 3.
7. The pluripotent stem cell according to embodiment 6, wherein the construct comprises the sequence of SEQ ID NO: 4.
8. The pluripotent stem cell according to any one of embodiments 1-7, wherein the activity of the transcriptional regulator protein is controlled by an exogenously supplied substance derivative.
9. The pluripotent stem cell according to any one of embodiments 1-8, wherein the transcriptional regulator protein is selected from the group consisting of: tetracycline responsive transcriptional activator protein (rtTa), Tetracycline repressor (TetR), VgEcR synthetic receptor or a hybrid transcriptional regulator protein comprising a DNA binding domain from the yeast GAL4 protein, a truncated ligand binding domain from the human progesterone receptor and an activation domain from the human NF-kB, preferably the transcriptional regulator protein is rtTA.
10. The pluripotent stem cell according to any one of embodiments 1-9, wherein the inducible promoter includes a Tet Responsive Element (TRE).
11. The pluripotent stem cell according to any one of embodiments 1-10, wherein the inducible promotor is a tetON promotor.
12. The pluripotent stem cell according to any one of embodiments 1-11, wherein said first and further genomic safe harbour sites are selected from any two of the hROSA26 locus, the AAVS1 locus, the CLYBL gene or the CCR5 gene, preferably wherein the genetic safe harbour site are hROSA26 locus and the AAVS1 locus.
13. A method for the production of adipocytes, preferably white adipocytes, comprising
a) culturing the pluripotent stem cell according to any one of the preceding claims in a proliferation medium: followed by
b) inducing adipocyte differentiation by adding the exogenous substance according to claim 8, preferably the proliferation and/or differentiation medium does not comprise insulin and/or dexamethasone.
14. The method according to embodiment 13, wherein the differentiation phase is at most 10 days, at most 9 days, 8 days, 7 days, 6 days, 5 days, 4 days or 3 days.
15. The method according to embodiment 13 or 14 wherein the produced adipocytes are for human and non-human dietary consumption.
16. Use of a pluripotent stem cell according to any one of embodiments 1-12 or use of the method for producing a adipocyte according to any one of embodiments 13-15 for tissue engineering, optionally for the production of cultured meat.
17. A food product comprising the pluripotent stem cell according to any one of embodiments 1-12 or the adipocytes obtained by the method according to any one of claims 13-15.
18. The food product according to embodiment 17, wherein the food product is cultured meat.

1. A pluripotent stem cell comprising:
i) an expression construct for expression of a transcriptional regulator protein inserted into a first genetic safe harbour site;
ii) an expression construct for expression of a PPAR-γ protein, wherein the coding sequence for the PPAR-γ protein is operably linked to an inducible promoter; and,
iii) an expression construct for expression of a CEBPα protein, wherein the coding sequence for the CEBPα protein is operably linked to an inducible promoter;
wherein the expression constructs of ii) and iii) are inserted into at least one further genetic safe harbour site that is not the first genetic safe harbour site,
and wherein the inducible promotor is regulated by the transcriptional regulator protein.

2. A pluripotent stem cell according to claim 1, wherein the expression constructs of ii) and iii) are both inserted into a second genetic safe harbour site that is different from the first genetic safe harbour site.

3. The pluripotent stem cell according to claim 1, wherein the cell is selected from the group consisting of induced pluripotent stem cells, and embryonic cell lines.

4. The pluripotent stem cell according to claim 1, wherein the pluripotent stem cells are of a livestock or poultry species.

5. The pluripotent stem cell according to claim 4, wherein the livestock species is porcine or bovine.

6. The pluripotent stem cell according to claim 2, wherein the expression construct that is inserted into the second genetic safe harbour site encodes a PPAR-γ protein a linker and a CEBPα protein.

7. The pluripotent stem cell according to claim 6, wherein the construct comprises the sequence of SEQ ID NO: 4.

8. The pluripotent stem cell according to claim 1, wherein the activity of the transcriptional regulator protein is controlled by an exogenously supplied substance derivative.

9. The pluripotent stem cell according to claim 1, wherein the transcriptional regulator protein is selected from the group consisting of: tetracycline responsive transcriptional activator protein (rtTa), Tetracycline repressor (TetR), VgEcR synthetic receptor or a hybrid transcriptional regulator protein comprising a DNA binding domain from the yeast GAL4 protein, a truncated ligand binding domain from the human progesterone receptor and an activation domain from the human NF-kB, preferably the transcriptional regulator protein is rtTA.

10. The pluripotent stem cell according to claim 1, wherein the inducible promoter includes a Tet Responsive Element (TRE).

11. The pluripotent stem cell according to claim 1, wherein the inducible promotor is a tetON promotor.

12. The pluripotent stem cell according to claim 1, wherein said first and further genomic safe harbour sites are selected from any two of the hROSA26 locus, the AAVS1 locus, the CLYBL gene or the CCR5 gene.

13. A method for the production of adipocytes, comprising
a) culturing the pluripotent stem cell according to claim 1 in a proliferation medium: followed by
b) inducing adipocyte differentiation by adding an exogenous substance, preferably the proliferation and/or differentiation medium does not comprise insulin and/or dexamethasone.

14. The method according to claim 13, wherein the differentiation phase is at most 10 days, at most 9 days, 8 days, 7 days, 6 days, 5 days, 4 days or 3 days.

15. The method according to claim 13 wherein the produced adipocytes are for human and non-human dietary consumption.

16. (canceled)

17. A food product comprising the pluripotent stem cell according to claim 1.

18. The food product according to claim 17, wherein the food product is cultured meat.
